(19) United States
(12) Patent Application Publication
(10) Pub. No.: US 2007/0154470 A1
(43) Pub. Date: Jul. 5, 2007

(54) Holt's O(log N) method to search a massively poly-clonal collection of hybridomas to detect, select and isolate target-antigen-specific high-affinity monoclonal-antibody-generating hybridomas for the rapid development and manufacture of highly specific and effectively targeted monoclonal antibodies

(76) Inventor: Gerhard B. Holt, Sydney, NY (US)

Correspondence Address: GERHARD B. HOLT, #129, Wentworth Bldg - University of Sydney, Sydney / Camperdown, 2006, AU

(21) Appl. No.: 11/320,295

(22) Filed: Dec. 29, 2005

(51) Int. Cl. A61K 39/395; C07K 16/18; G06F 19/00; C12N 5/06

(52) U.S. Cl. 424/133.1; 530/387.1; 702/19; 435/326

(57) ABSTRACT

This invention is an O(Log N) technique that searches a massively poly-clonal collection of hybridomas (or other monoclonal antibody generators) to detect, select and isolate target-antigen-specific high-affinity monoclonal-antibody-generating hybridomas (or other monoclonal antibody generators) for the rapid development and manufacture of highly specific and effectively targeted monoclonal antibodies. It expedites the manufacture of monoclonal antibodies targeted at a specific antigen or collection of antigens—making the generation of “poly-monoclonal” antibody “cocktails” more practical and efficient.

### KEY RESOURCES USED INCLUDE

Target antigen or antigens (for example—but not by way of limitation—tumor surface antigens, tumor associated antigens, bacterial surface antigens, receptors, signaling chemical messengers (for example—but not by way of limitation—cytokines, chemokines, paracrine or endocrine related chemicals) etc).

Strips Containing Target Antigen or Antigens.

(Refer to this as a “target-antigen-expressing strip”).

Polyclonal Hybridomas.

(These could be generated using standard techniques, and could—by way of example but not by way of limitation—also involve the use of xeno-immunization approaches or pooling of many allo-donor samples).

Containers for Storing and Processing the Hybridomas in the Presence of the Target Antigens.

Start with k containers.

For illustration and discussion purposes k will be set to 2 here (but not by way of limitation—k can be a larger number of containers).

For purposes of our discussion, container #1 will be referred to as container[1], and container #2 will be referred to as container[2].

### METHOD

Step 1: Isolate/Purify the Current Hybridoma Sample.

Separate the poly-clonal hybridomas from their mixture of poly-monoclonal antibodies. Standard techniques can be used for this purpose. Hybridomas are very different in weight, volume and various other physical and chemical attributes than the monoclonal antibodies that they generate. (For example—but not by way of limitation—cell sorters, and/or various chromatography approaches could be used).

Step 2: Divide the Current Hybridoma Sample into k Parts, and Generate New Monoclonal Antibodies in Each of these K Sub-Samples.

Divide the current mixture of hybridomas into k portions, and put each portion into one of the k labeled containers. (For example, if k is set to 2, divide the current mixture of hybridomas into 2 portions, put one half into container[1], and the other half into container[2]). Add appropriate materials to sustain (but not expand) the hybridomas and to allow for production of a polyclonal mixture of monoclonal antibodies. At the end of this step, each container will contain a mixture of hybridomas, and of *newly* generated monoclonal antibodies derived from these hybridomas.

Step 3: Test the k Subsamples for the Presence of any Target-Antigen Binding Monoclonal Antibody that was Newly Generated in that Subsample.

Add a “target-antigen-expressing strip” to each of the k containers.

(For example, if k is set to 2, put one “target-antigen-expressing strip” into container[1], and another “target-antigen-expressing strip” into container[2]).

If any of the newly generated monoclonal antibodies from a given container[i] binds the target antigen, it will bind with the strip. Standard Eliza-like techniques can be used to detect the presence of the monoclonal antibody bound to target antigen on the “target-antigen-expressing strip”.

Note:

IF the monoclonal antibody IS detected on a “target-antigen-expressing strip” in a given container[i] this means that we have successfully found a monoclonal antibody against a desired target antigen. FURTHERMORE, we ALSO know that the given container[i] in question contains a HYBRIDOMA that GENERATED this monoclonal antibody, which now we will proceed in subsequent steps to isolate and expand.

Efficiently locating and isolating a HYBRIDOMA that generates monoclonal antibodies versus a desired target antigen from a massively polyclonal pre-existing pool of hybridomas is an important contribution of this invention.

Step 4: Repeat these Processes to Isolate and Purify a “Useful” Target Hybridoma that Generates a Desired (Target Antigen Binding) Monoclonal Antibody.

Repeat STEPS 1 though 3 on the hybridomas in the “positive” container[i].

(Hybridomas in the other containers could be saved for subsequent use).

Step 4: (Continued)

For example, if k was set to 2, and container[1] was found to contain a monoclonal antibody that bound to the “target-antigen-expressing strip”, we will now repeat STEPS 1 through 3 using ONLY the hybridomas found in container[1]. In other words, we will separate out the hybridomas (STEP 1), divide the hybridomas in to k containers (in this example 2 containers) (STEP 2), and add new copies of the “target-antigen-expressing strip” to each of these containers (STEP 3) to see which portion (in this example—which one of the two portions) of the hybridomas contain a “useful” target hybridoma (a hybridoma that generates a monoclonal antibody that binds the “target-antigen-expressing strip”).

By repeating these steps several times we will rapidly converge on the desired hybridoma by a process that is O(Log N). In other words, if we repeat the process N times (with k set to 2), we can effectively search through 2 raised to the Nth power different hybridomas to find a single hybridoma that generates a monoclonal antibody that binds to the target antigen in question. At the end of these N repetitions, we will have isolated that desired hybridoma that we can then expand using standard techniques, and use to manufacture copious quantities of the desired monoclonal antibody using standard techniques.

For example—but not by way of limitation—repeating the process a mere 30 times with k set to 2 containers would allow us to search through 2ˆ30 (over 1 billion) hybridomas to find and isolate a single “useful” target hybridoma.

(Note that this method is somewhat related to, but a substantial distinct improvement upon standard limiting dilution techniques, and allows for very rapid search of massively polyclonal pools of hybridomas to detect “useful” target hybridomas. Because the process is O(Log N), a few additional repetitions will allow for searches through even more widely diverse pools of hybridomas, and will allow rapid purification/isolation of the desired target(s).)

(Note also, that if more than one container contains a “useful” target hybridoma, then the process can continue on ANY of the “positive” containers. The hybridomas in the other “positive” containers could either be discarded, saved individually for further search and use, or even mixed together for further search and use.)

1: Poly-monoclonal antibody generation, detection, selection, isolation, and/or use.
This invention allows for the generation, detection, selection, isolation, and/or use of several or many (3 or more) DIFFERENT monoclonal antibodies to target a given antigen (or antigens).

2: O(Log N) method to efficiently manufacture poly-monoclonal antibody generators targeted to a particular antigen or collection of antigens.
This invention provides an O(Log N) method to search a massively poly-clonal collection of monoclonal antibody generators (typically (but not by way of limitation) hybridomas) to allow for efficient detection, selection, and isolation of target-antigen-specific high-affinity monoclonal-antibody-generators (typically (but not by way of limitation) hybridomas) for the rapid development and manufacture of highly specific and effectively targeted monoclonal antibodies.

3: This invention efficiently locates and isolates a HYBRIDOMA that generates monoclonal antibodies versus a desired target antigen from a massively polyclonal pre-existing pool of hybridomas. Once isolated, this hybridoma can be used to readily manufacture substantial quantities of its monoclonal antibody. By dramatically facilitating the efficiency of the location and isolation process to be O(Log N), this invention readily allows for the practical generation and use of multiple (3 or more (but potentially 10 s or 100 s of)) different monoclonal antibodies targeted at a chosen antigen (or collection of antigens)—creating a “poly-monoclonal” antibody “cocktail” that can more reliably and effectively bind the desired target antigen (or collection of antigens).
